(19) United States
(12) Patent Application Publication
(10) Pub. No.: US 2011/0021532 A1
(43) Pub. Date: Jan. 27, 2011

(54) NOVEL SUBSTITUTED HETEROAROMATIC COMPOUNDS AS INHIBITORS OF STEAROYL-COENZYME A DELTA-9 DESATURASE

(75) Inventors: David Powell, Verdun (CA); Marie-Eve Lebrun, Dollard-des-Ormeaux (CA); Sathesh Bhat, Dollard-des-Ormeaux (CA); Yeeman K. Ramtohul, Pierrefonds (CA)

Correspondence Address: MERCK, P O BOX 2000, RAHWAY, NJ, 07065-0907, US

(73) Assignee: Merck Frosst Canada Ltd.

(21) Appl. No.: 12/934,100

(22) PCT Filed: Apr. 21, 2009
(86) PCT No.: PCT/CA09/00541

§ 371 Date: Sep. 23, 2010

Related U.S. Application Data
(60) Provisional application No. 61/125,065, filed on Apr. 22, 2008.

(51) Int. Cl. A61K 31/501; C07D 235/18; A61K 31/4184; C07D 401/02; A61K 31/4439; C07D 403/04; A61K 31/506; C07D 471/02; A61K 31/437; A61P 3/10; A61P 3/00; A61P 3/04; A61P 1/16; A61P 9/00; A61P 9/10

(52) U.S. Cl. 514/252.06; 548/310.7; 514/394; 548/304.7; 546/273.4; 514/338; 544/238; 544/333; 514/256; 546/121; 514/300

(57) ABSTRACT

Substituted heteroaromatic compounds of structural formula I are inhibitors of stearoyl-coenzyme A delta-9 desaturase. The compounds of the present invention are useful for the prevention and treatment of conditions related to abnormal lipid synthesis and metabolism, including cardiovascular disease, such as atherosclerosis; obesity; Type 2 diabetes; insulin resistance; hyperglycemia; Metabolic Syndrome; neurological disease; cancer; and liver steatosis. Formula (I).

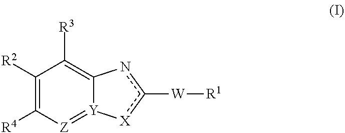

### FIELD OF THE INVENTION

The present invention relates to novel substituted heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease, such as atherosclerosis; obesity; Type 2 diabetes; insulin resistance; hyperglycemia; Metabolic Syndrome; neurological disease; cancer; and hepatic steatosis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). Elevated SCD activity in adipose tissue is closely coupled to the development of insulin resistance (Sjogren, et al., Diabetologia, 51(2): 328-35 (2007)). The postprandial de novo lipogenesis is significantly elevated in obese subjects (Marques-Lopes, et al., American Journal of Clinical Nutrition, 73: 252-261 (2001)). Knockout of the SCD gene ameliorates Metabolic Syndrome by reducing plasma triglycerides, reducing weight gain, increasing insulin sensitivity, and reduces hepatic lipid accumulation (MacDonald, et al., Journal of Lipid Research, 49(1): 217-29 (2007)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)). RNA interference of SCD-1 reduces human tumor cell survival (Morgan-Lappe, et al., Cancer Research, 67(9): 4390-4398 (2007)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications WO 2005/011653, WO 2005/011654, WO 2005/011656, WO 2005/011656, WO 2005/011657, WO 2006/014168, WO 2006/034279, WO 2006/034312, WO 2006/034315, WO 2006/034338, WO 2006/034341, WO 2006/034440, WO 2006/034441, WO 2006/034446, WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; WO 2007/136746; WO 2008/036715; WO 2008/074835; WO 2008/127349; and U.S. Pat. Nos. 7,456,180 and 7,390,813; all assigned to Xenon Pharmaceuticals, Inc. or Xenon Pharmaceuticals, Inc./Novartis AG.

A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/056846 (24 May 2007); WO 2007/071023 (28 Jun. 2007); WO 2007/134457 (29 Nov. 2007); WO 2007/143823 (21 Dec. 2007); WO 2007/143824 (21 Dec. 2007); WO 2008/017161 (14 Feb. 2008); WO 2008/046226 (24 Apr. 2008); WO 2008/064474 (5 Jun. 2008); WO 2008/089580 (31 Jul. 2008); WO 2008/128335 (30 Oct. 2008); WO 2008/141455 (27 Nov. 2008); US 2008/0132542 (5 Jun. 2008); US 2008/0182838 (31 Jul. 2008); and WO 2009/012573 (29 Jan. 2009).

WO 2008/003753 (assigned to Novartis) discloses a series of pyrazolo[1,5-α]pyrimidine analogs as SCD inhibitors; WO 2007/143597 and WO 2008/024390 (assigned to Novartis AG and Xenon Pharmaceuticals) disclose heterocyclic derivatives as SCD inhibitors; and WO 2008/096746 (assigned to Takeda Pharmaceutical) disclose spiro compounds as SCD inhibitors.

Additional international patent applications disclosing SCD inhibitors have published: WO 2008/062276 (Glenmark; 29 May 2008); WO 2008 (Glenmark; 13 Mar. 2008); WO 2008/003753 (Biovitrum AB; 10 Jan. 2008); WO 2008/135141 (Sanofi-Aventis; 13 Nov. 2008); WO 2008/157844 (Sanofi-Aventis; 24 Dec. 2008); WO 2008/104524 (SKB; 4 Sep. 2008); WO 2008/074834 (SKB; 26 Jun. 2008); WO 2008/074833 (SKB; 26 Jun. 2008); WO 2008/074832 (SKB; 26 Jun. 2008); WO 2008/074824 (SKB; 26 Jun. 2008); WO 2009/0010560 (SKB; 22 Jan. 2009); WO 2009/016216 (SKB; 5 Feb. 2009); and WO 2008/139845 (Daiichi-Sankyo; 20 Nov. 2008)

Small molecule SCD inhibitors have also been described by (a) G. Liu, et al., “Discovery of Potent, Selective, Orally Bioavailable SCD1 Inhibitors,” in J. Med. Chem., 50: 3086-3100 (2007); (b) H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007); and (c) Z. Xin, et al., “Discovery of piperidine-aryl urea-based stearoyl-CoA desaturase 1 inhibitors,” Bioorg. Med. Chem. Lett., 18: 4298-4302 (2008).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobrzyn and J. M. Ntambi, in “Stearoyl-CoA desaturase as a new drug target for obesity treatment,” Obesity Reviews, 6: 169-174 (2005).

### SUMMARY OF THE INVENTION

The present invention relates to substituted heteroaromatic compounds of structural formula I:

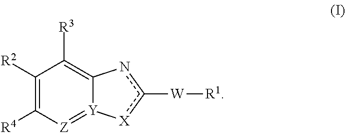

These substituted heteroaromatic compounds are effective as inhibitors of SCD. They are therefore useful for the treatment, control or prevention of disorders responsive to the inhibition of SCD, such as Type 2 diabetes, insulin resistance, hyperglycemia, lipid disorders, obesity, atherosclerosis, and Metabolic Syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, hyperglycemia, obesity, lipid disorders, atherosclerosis, and Metabolic Syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating Metabolic Syndrome by administering the compounds of the present invention in combination with a therapeutically effective amount of another agent known to be useful to treat the condition.

### DETAILED DESCRIPTION OF THE INVENTION

The present invention is concerned with novel substituted heteroaromatic compounds useful as inhibitors of SCD. Compounds of the present invention are described by structural formula I:

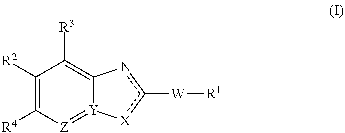

and pharmaceutically acceptable salts thereof; wherein
X is NH, Y is C, and Z is N or CR⁵;
or X and Z are each CR⁵, and Y is N;
W is a residue selected from the group consisting of:

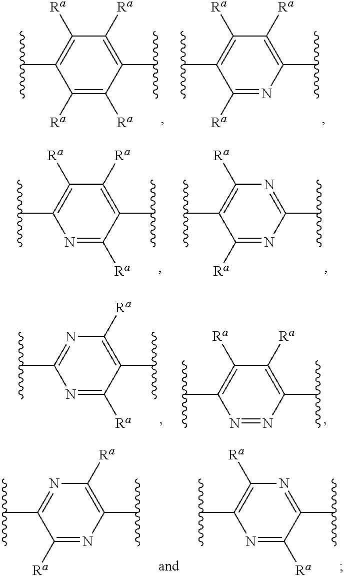

wherein each R^{a} is independently selected from the group consisting of:

hydrogen,

halogen,

C₁₋₄ alkyl, optionally substituted with one to five fluorines, and

C₁₋₄ alkoxy, optionally substituted with one to five fluorines;
R³, R⁴, and each R⁵ are each independently selected from the group consisting of:

hydrogen,

halogen,

C₁₋₄ alkyl, optionally substituted with one to five fluorines, and

C₁₋₄ alkoxy, optionally substituted with one to five fluorines;
R² is selected from the group consisting of:

—SO₂cyclopropyl,

—SC₁₋₃ alkyl, optionally substituted with one to five fluorines,

—S(O)C₁₋₃ alkyl, optionally substituted with one to five fluorines,

—SO₂C₁₋₃ alkyl, optionally substituted with one to five fluorines, and

—SO₂NR^{b}R^{b}, wherein each R^{b} is independently hydrogen or C₁₋₃ alkyl; and
R¹ is selected from the group consisting of:

cyclopentenyl,

cyclohexenyl,

phenyl, and

heteroaryl selected from the group consisting of:
pyridyl,
pyrimidinyl,
pyridazinyl,
pyrazinyl,
furyl,
thienyl,
thiazolyl,
oxazolyl,
isoxazolyl,
isothiazolyl,
imidazolyl, and
pyrazolyl;
wherein aryl and heteroaryl are optionally substituted with one to three substituents independently selected from the group consisting of halogen, hydroxy, cyano, and C₁₋₃ alkyl
wherein alkyl is optionally substituted with one to five fluorines.

In one embodiment of the compounds of the present invention, X is NH, Y is C, and Z is N or CR⁵; as described by structural formula (Ia):

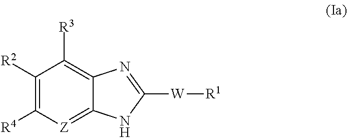

and pharmaceutically acceptable salts thereof; wherein Z, R¹, R², R³, and R⁴ are as described above.

In a class of this embodiment, Z is CR⁵, and R² is —SO₂CH₃ or —SO₂cyclopropyl.

In a second class of this embodiment, Z is CH, and R³ and R⁴ are each hydrogen. In a subclass of this second class of this embodiment, R² is —SO₂CH₃ or —SO₂cyclopropyl.

In a third class of this embodiment, W is

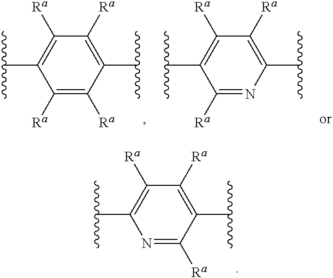

In a subclass of this third class of this embodiment, each R^{a} is hydrogen. In another subclass of this third class of this embodiment, W is

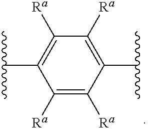

In a subclass of this subclass, each R^{a} is hydrogen.

In a fourth class of this embodiment, R¹ is phenyl optionally substituted with one to two halogens selected from fluorine and chlorine.

In a fifth class of this embodiment,
Z is CR⁵;
W is

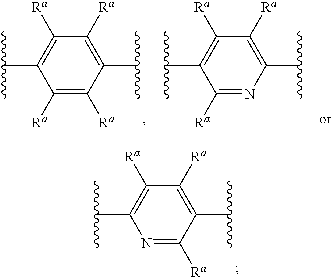

R¹ is phenyl optionally substituted with one to two halogens selected from chlorine and fluorine; and
R² is —SO₂CH₃ or —SO₂cyclopropyl;
wherein R^{a} and R⁵ are as defined above.

In a subclass of this fifth class of this embodiment, R^{a}, R³, R⁴, and R⁵ are each hydrogen.

In a second embodiment of the compounds of the present invention, X and Z are each CR⁵, and Y is N; as described in structural formula (Ib):

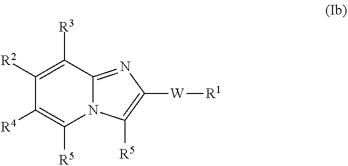

and pharmaceutically acceptable salts thereof; wherein R¹, R², R³, R⁴, and R⁵ are as described above.

In a class of this second embodiment, R² is —SO₂CH₃ or —SO₂cyclopropyl.

In a second class of this second embodiment, R³, R⁴, and each R⁵ are each hydrogen. In a subclass of this second class of this embodiment, R² is —SO₂CH₃ or —SO₂cyclopropyl.

In a third class of this second embodiment, W is

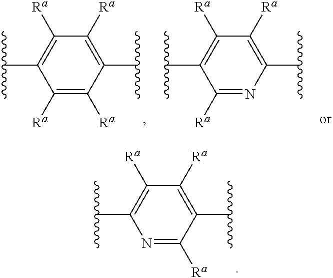

In a subclass of this third class of this second embodiment, each R^{a} is hydrogen. In another subclass of this third class of this second embodiment, W is

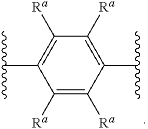

In a subclass of this subclass, each R^{a} is hydrogen.

In a fourth class of this second embodiment, R¹ is phenyl optionally substituted with one to two halogens selected from chlorine and fluorine.

In a fifth class of this second embodiment,
W is

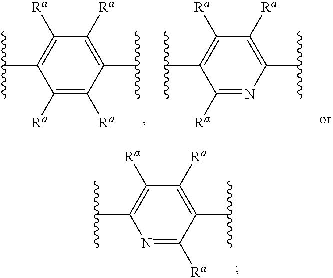

R¹ is phenyl optionally substituted with one to two halogens selected from chlorine and fluorine; and
R² is —SO₂CH₃ or —SO₂cyclopropyl;
wherein R^{a} is as defined above.

In a subclass of this fifth class of this second embodiment, R^{a}, R³, R⁴, and each R⁵ are each hydrogen.

Illustrative, but nonlimiting, examples of compounds of the present invention that are useful as inhibitors of SCD are the following:

| Example | IC₅₀ hSCD-1 |
|---|---|
| 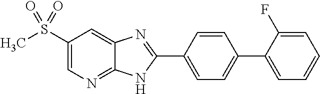 | 113 nM |
| 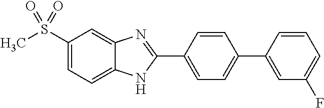 | 26 nM |
| 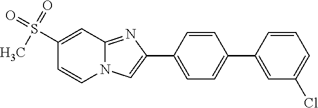 | 13 nM |
| 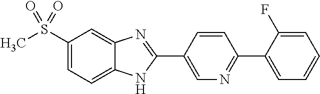 | 27 nM |
| 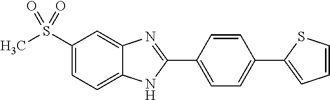 | 231 nM |

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. When no number of carbon atoms is specified, C₁₋₆ is intended.

“Cycloalkyl” means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” shall mean straight or branched-chain alkenes having the specified number of carbon atoms. Examples of alkenyl include vinyl, 1-propenyl, 1-butenyl, 2-butenyl, and the like.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention. Examples of tautomers which are intended to be encompassed within the compounds of the present invention are illustrated below:

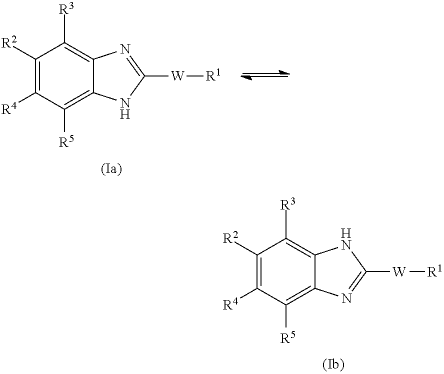

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, and other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD Enzyme Activity Assay:

The potency of compounds of formula I against the stearoyl-CoA desaturase was determined by measuring the conversion of radiolabeled stearoyl-CoA to oleoyl-CoA using rat liver microsome or human SCD1 (hSCD-1) following previously published procedures with some modifications (Joshi, et al., J. Lipid Res., 18: 32-36 (1977); Talmo, et al., Anal. Biochem, 29: 300-304 (1969)). Liver microsome was prepared from male Wistar or Sprague Dawley rats on a high carbohydrate diet for 3 days (LabDiet #5803, Purina). The livers were homogenized (1:10 w/v) in a buffer containing 250 mM sucrose, 1 mM EDTA, 5 mM DTT and 50 mM Tris-HCl (pH 7.5). After a 100,000×g centrifugation for 60 min, the liver microsome pellet was suspended in a buffer containing 100 mM sodium phosphate, 20% glycerol, 2 mM DTT, and stored at −78° C. Human SCD1 desaturase system was reconstituted using human SCD1 from a baculovirus/Sf9 expression system, cytochrome B5 and cytochrome B5 reductase. Typically, test compound in 2 μL DMSO was incubated for 15 min at room temperature with 180 μL of the SCD enzyme in a buffer containing 100 mM Tris-HCl (pH 7.5), ATP (5 mM), Coenzyme-A (0.1 mM), Triton X-100 (0.5 mM) and NADH (2 mM). The reaction was initiated by the addition of 20 μL of [³H]-stearoyl-CoA (final concentration=2 μM, radioactivity concentration=1 μCi/mL). After 10 min, the reaction mixture (80 μL) was mixed with a calcium chloride/charcoal aqueous suspension (100 μL charcoal (10% w/v) plus 25 μL CaCl₂ (2N). After centrifugation to precipitate the radioactive fatty acid species, tritiated water released from 9,10-[³H]-stearoyl-CoA by the SCD enzyme was quantified on a scintillation counter.

### II. Whole Cell-Based SCD (Delta-9), Delta-5 and Delta-6 Desaturase Assays:

Human HepG2 cells were grown on 96-well plates in MEM media (Gibco cat #11095-072) supplemented with 10% heat-inactivated fetal bovine serum at 37° C. under 5% CO₂ in a humidified incubator. Test compound dissolved in the media was incubated with the sub-confluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of formula I, particularly the inhibitors of Examples 1 to 30, exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 to 30, is at least about ten or more, and preferably about one hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefore, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); vildagliptin (LAF 237); denagliptin; P93/01; saxagliptin (BMS 477118); RO0730699; MP513; SYR-322: ABT-279; PHX1149; GRC-8200; and TS021.

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB 1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. No. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:
(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g.

troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119; and
(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention:

The compounds of structural formula I can be prepared according to the procedures of the following schemes and examples, using appropriate materials and are further exemplified by the following specific examples. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESI). ¹H NMR were recorded on a Bruker instrument at 400 MHz.

### List of Abbreviations:

Alk=alkyl
Ar=aryl
br=broad
CH₂Cl₂=dichloromethane
d=doublet
DBU=1,8-diazabicyclo[5.4.0]undec-7-ene
DMF=dimethylformamide
DMSO=dimethylsulfoxide
dppf=(diphenylphosphino)ferrocene
ESI=electrospray ionization
EtOAc=ethyl acetate
HOAc=acetic acid
KOH=potassium hydroxide
LiOH=lithium hydroxide
m=multiplet
MeCN=Acetonitrile
MeOH=methyl alcohol
MgSO₄=magnesium sulfate
MS=mass spectroscopy
NaOH=sodium hydroxide
Na₂SO₄=sodium sulfate
NMR=nuclear magnetic resonance spectroscopy
PG=protecting group
rt=room temperature
s=singlet
t=triplet
THF=tetrahydrofuran
TMSCl=chlorotrimethylsilane
TFA=trifluoroacetic acid
p-TsOH=toluene-4-sulfonic acid

The following Examples are provided to illustrate the invention and are not to be construed as limiting the scope of the invention in any manner.

### Method A:

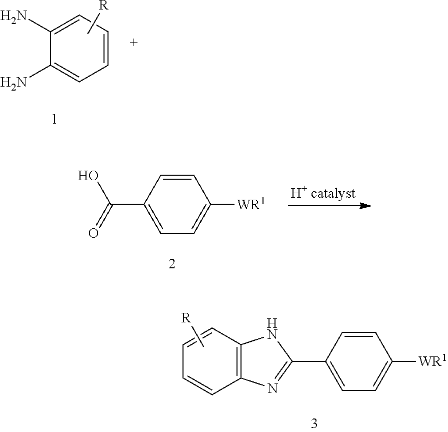

The appropriately substituted diamine 1 is heated in the presence of carboxylic acid 2 and a catalytic amount of a protic acid (such as polyphosphoric acid, sulfuric acid, and hydrochloric acid) at elevated temperatures (50-150° C.) to generate the benzimidazoles 3.

### Method B:

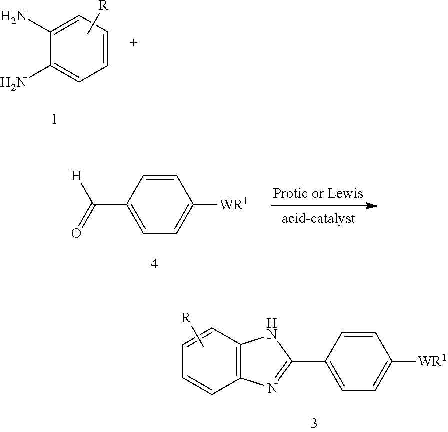

The appropriately substituted diamine 1 is heated in the presence of an aldehyde 4 and a catalytic amound of a protic acid (such as para-toluenesulfonic acid) or Lewis acid (such as chlorotrimethylsilane) at elevated temperatures (50-150° C.), in an appropriate dipolar aprotic solvent (such as DMF, DMA, and DMSO) to generate the benzimidazoles 3.

### Method C:

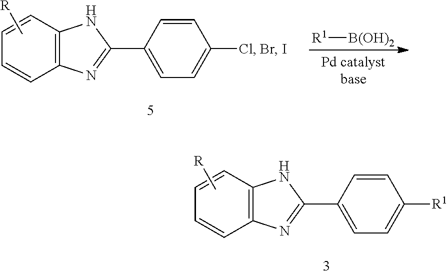

A mixture of arylhalide 5 is heated with a boronic acid or boronate in the presence of a suitable palladium catalyst (such as Pd₂dba₃, PdCl₂, Pd(OAc)₂ and [(allyl)PdCl]₂), a phosphine ligand (such as PPh₃, PCy₃, P(t-Bu)₃, and biphenylP(Cy)₂) and a base (such as K₃PO₄, Cs₂CO₃, CsF and KOt-Bu) in a polar solvent to provide the cross-coupled product 3.

### Method D:

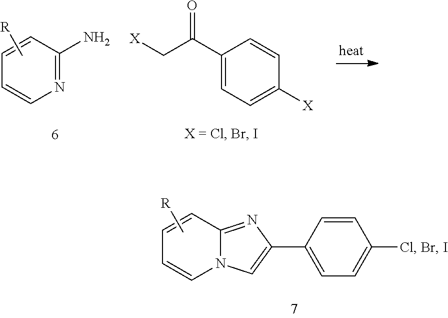

A solution of a 2-aminopyridine 6 is heated in the presence of an alpha-halo ketone to afford the imidazolo[1,2-α]pyridine 7.

### Method E:

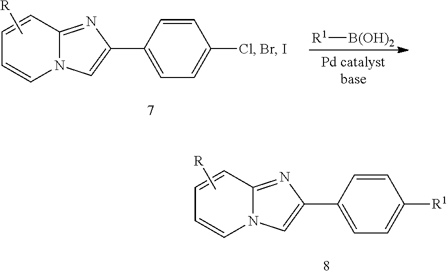

A mixture of aryl halide 7 is heated with a boronic acid or boronate in the presence of a suitable palladium catalyst (such as Pd₂dba₃, PdCl₂, Pd(OAc)₂ and [(allyl)PdCl]₂), a phosphine ligand (such as PPh₃, PCy₃, P(t-Bu)₃, and biphenylP(Cy)₂) and a base (such as K₃PO₄, Cs₂CO₃, CsF and KOt-Bu) in a polar solvent to provide the cross-coupled product 8.

### Example 1

### 2-(2′-Fluorobiphenyl-4-yl-6-(methylsulfonyl)-1H-benzimidazole

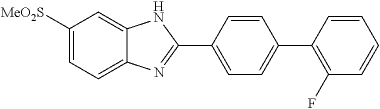

### Step 1: 2′-Fluorobiphenyl-4-carbaldehyde

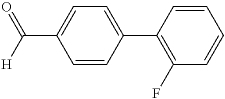

Into a 100 mL pressure flask equipped with a magnetic stirbar and under nitrogen was added 2-fluoroboronic acid (4.76 g, 34.1 mmol), 4-bromobenzaldehyde (5.25 g, 28.4 mmol), PdCl₂(dppf) (1.16 g, 1.42 mmol) and DMF (40 mL). The solution was treated with 2 M aqueous sodium carbonate solution (28 mL, 56.8 mmol) and the vial was sealed and heated to 90° C. for 16 h. The cooled mixture was poured into a 250 mL separatory funnel containing water (125 mL) and the mixture extracted with ethyl acetate (3×50 mL). The combined organic layers were washed with brine, dried over MgSO₄, filtered and concentrated. Purification by column chromatography on silica gel, eluting with 0% EtOAc in hexanes to 20% EtOAc in hexanes as a gradient, gave the title compound as a solid.

### Step 2: 6-Bromo-2-(2′-fluorobiphenyl-4-yl)-1H-benzimidazole

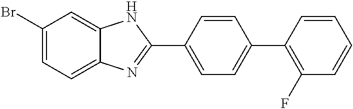

Into a 5 mL microwave flask, equipped with a magnetic stirbar was added 4-bromobenzene-1,2-diamine (200 mg, 1.07 mmol), p-toluenesulfonic acid monohydrate (20 mg, 0.11 mmol) and 2′-fluorobiphenyl-4-carbaldehyde (214 mg, 1.07 mmol) in DMF (2 mL). The vial was sealed and heated to 100° C. under microwave irradiation. The cooled mixture was poured into a 125 mL separatory funnel containing water (75 mL) and the mixture extracted with ethyl acetate (3×30 mL). The combined organic layers were washed with brine, dried over MgSO₄, filtered and concentrated. Purification by column chromatography on silica gel eluting with 10% MeCN in toluene gave the title compound as a white solid.

MS (ESI, Q⁺) m/z 367, 369 (M+1).

### Step 3: 2-(2′-Fluorobiphenyl-4-yl)-6-(methylsulfonyl)-1H-benzimidazole

Into a 10 mL sealable pressure flask, equipped with a magnetic stirbar and under nitrogen was added 6-bromo-2-(2′-fluorobiphenyl-4-yl)-1H-benzimidazole (100 mg, 0.272 mmol), sodium methanesulfinate (56 mg, 0.545 mmol), copper(I) trifluoromethanesulfonate benzene complex (137 mg, 0.272 mmol) and trans-1,2-diaminocyclohexane (65 μL, 0.545 mmol) in DMSO (2 mL). The contents of the flask were purged under nitrogen for 10 min, and the resulting suspension was heated to 130° C. for 6 h. The mixture was cooled to room temperature and poured into a 125 mL separatory funnel containing water (75 mL) and the mixture extracted with ethyl acetate (3×30 mL). The combined organic layers were washed with brine, dried over MgSO₄, filtered and concentrated. Purification by column chromatography on silica gel, eluting with 20% EtOAc in hexanes to 50% EtOAc in hexanes, gave the title compound as a solid.

¹H NMR (400 MHz, d₆-acetone): δ 8.39-8.37 (2H, m), 8.23 (1H, s), 7.84-7.81 (4H, m), 7.67-7.63 (1H, m), 7.50-7.46 (1H, m), 7.38-7.30 (2H, m), 3.17 (3H, s). MS (ESI, Q⁺) m/z 367 (M+1).

### Example 2

### 6-(Cyclopropylsulfonyl)-2-(2′-fluorobiphenyl-4-yl)-1)-1H-benzimidazole

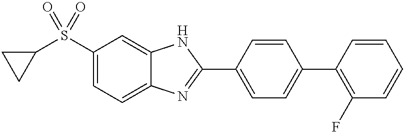

Into a 10 mL sealable pressure flask, equipped with a magnetic stirbar and under nitrogen was added 6-bromo-2-(2′-fluorobiphenyl-4-yl)-1H-benzimidazole (100 mg, 0.272 mmol), sodium cyclopropylsulfinate (69 mg, 0.545 mmol), copper(I) trifluoromethanesulfonate benzene complex (137 mg, 0.272 mmol) and trans-1,2-diaminocyclohexane (65 μL, 0.545 mmol) in DMSO (2 mL). The contents of the flask were purged under nitrogen for 10 min, and the resulting suspension was heated to 130° C. for 6 h. The mixture was cooled to room temperature, and poured into a 125 mL separatory funnel containing water (75 mL) and the mixture extracted with ethyl acetate (3×30 mL) The combined organic layers were washed with brine, dried over MgSO₄, filtered and concentrated. Purification by column chromatography on silica gel, eluting with 20% EtOAc in hexanes to 50% EtOAc in hexanes, gave the title compound as a solid.

¹H NMR (400 MHz, d₆-acetone): δ 8.40-8.38 (2H, m), 8.26-8.10 (2H, bm), 7.83-7.80 (3H, m), 7.68-7.64 (1H, m), 7.50-7.47 (1H, m), 7.38-7.29 (2H, m), 2.76-2.73 (1H, m), 1.24-1.21 (2H, m), 1.08-1.06 (2H, m). MS (ESI, Q⁺) m/z 393 (M+1).

### Example 3

### 2-Biphenyl-4-yl-6-(methylsulfonyl)-1H-benzimidazole

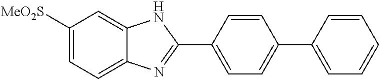

### Step 1: 4-(Methylsulfonyl)-2-nitroaniline

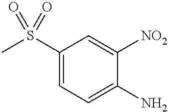

Into a 10 mL pressure flask equipped with a magnetic stirbar was added 4-fluoro-3-nitrophenyl methyl sulfone (2.5 g, 11.4 mmol) and methanol (2 mL). To this mixture was added concentrated aqueous ammonium hydroxide (2.0 mL) and the flask sealed and heated to 130° C. in an oil bath for 6 h. The resulting cooled yellow suspension was poured into water (100 mL) and filtered through Whatman #1 filter paper, washing with water. The title compound was isolated as a yellow solid which was dried under vacuum overnight.

MS (ESI, Q⁺) m/z 217 (M+1).

### Step 2: 4-(Methylsulfonyl)benzene-1,2-diamine

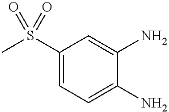

Into a 100 mL round-bottom flask equipped with a magnetic stirbar and reflux condenser was added 4-(methylsulfonyl)-2-nitroaniline (2.2 g, 10 mmol), and 5% Ru on carbon (100 mg) in ethanol (35 mL). Hydrazine hydrate (2.00 g, 41 mmol) was added and the reaction mixture was heated at reflux temperature for 1 h. The crude reaction mixture was cooled to room temperature and filtered through a pad of celite on a sintered glass funnel, washing with ethyl acetate. The filtrate was concentrated to give the desired product. MS (ESI, Q⁺) m/z 187 (M+1).

### Step 3: 2-(4-Bromophenyl)-6-(methylsulfonyl)-1H-benzimidazole

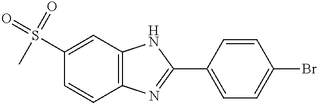

Into a 10 mL pressure flask equipped with a magnetic stirbar was added 4-(methylsulfonyl)benzene-1,2-diamine (2.50 g, 13.4 mmol), 4-bromobenzaldehyde (3.00 g, 16.1 mmol) and DMF (25 mL). The contents of the flask were treated with dropwise addition of TMSCl (4.3 mL, 33.6 mmol), the vial was sealed and heated in an oil bath to 90° C. for 4 h. The reaction mixture was cooled and poured into 100 mL of 2 M aqueous Na₂CO₃ solution. The resulting suspension was poured into a 250 mL separatory funnel containing water (50 mL) and the mixture was extracted with ethyl acetate (3×50 mL). The combined organic layers were washed with brine, dried over MgSO₄, filtered and the solvent was evaporated under reduced pressure. The product was purified by trituration with diethyl ether, and the resulting solid was collected by filtration through Whatman #1 filter paper, washed with diethyl ether and dried.

MS (ESI, Q⁺) m/z 351, 353 (M+1).

### Step 4: 2-Biphenyl-4-yl-6-(methylsulfonyl)-1H-benzimidazole

Into a 5 mL microwave vial equipped with a magnetic stirbar was added 2-(4-bromophenyl)-6-(methylsulfonyl)-1H-benzimidazole (70 mg, 0.20 mmol), phenylboronic acid (49 mg, 0.40 mmol), potassium phosphate, tribasic (106 mg, 0.50 mmol) and [PdBrP(t-Bu)₃]₂ (4 mg, 0.005 mmol). To these solids was added dioxane (2 mL) and water (0.1 mL), and the vial sealed and heated to 100° C. for 20 min. The reaction mixture was concentrated and purified by column chromatography on silica gel to give the desired product as a white solid.

MS (ESI, Q⁺) m/z 349 (M+1).

### Example 4

### 2-(4′-Fluorobiphenyl-4-yl)-6-(methylsulfonyl)-1H-benzimidazole

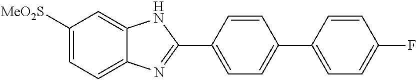

This compound was prepared from 2-(4-bromophenyl)-6-(methylsulfonyl)-1H-benzimidazole and 4-fluorophenylboronic acid according to the procedure outlined in Example 3, step 4.

¹H NMR (400 MHz, d₆-acetone): δ 8.34 (2H, d, J=8.0 Hz), 8.21 (1H, s), 7.90-7.78 (6H, m), 7.28 (2H, t, J=8.5 Hz), 3.14 (3H, s). MS (ESI, Q⁺) m/z 367 (M+1).

### Example 5

### 2-(3′-Fluorobiphenyl-4-yl)-6-(methylsulfonyl)-1H-benzimidazole

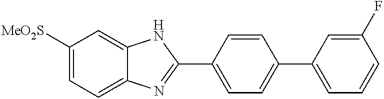

This compound was prepared from 2-(4-bromophenyl)-6-(methylsulfonyl)-1H-benzimidazole and 3-fluorophenylboronic acid according to the procedure outlined in Example 3, step 4.

¹H NMR (400 MHz, d₆-acetone): δ 8.37 (2H, d, J=8.0 Hz), 8.21 (1H, br s), 7.93 (2H, d, J=8.0 Hz), 7.82 (2H, s), 7.63-7.50 (3H, m), 7.19 (1H, t, 8.5 Hz), 3.14 (3H, s).

MS (ESI, Q⁺) m/z 367 (M+1).

### Example 6

### 2-(3′-Methylbiphenyl-4-yl)-6-(methylsulfonyl)-1H-benzimidazole

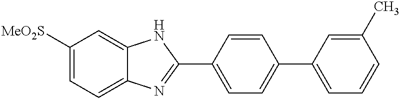

This compound was prepared from 2-(4-bromophenyl)-6-(methylsulfonyl)-1H-benzimidazole and 3-methylphenylboronic acid according to the procedure described in Example 3, step 4.

MS (ESI, Q⁺) m/z 363 (M+1).

### Example 7

### 2-(4-Cyclohex-1-en-1-ylphenyl-6-(methylsulfonyl)-1H-benzimidazole

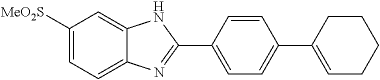

This compound was prepared from 2-(4-bromophenyl)-6-(methylsulfonyl)-1H-benzimidazole and cyclohex-1-en-1-yl boronic acid according to the procedure described in Example 3, step 4. MS (ESI, Q⁺) m/z 353 (M+1).

### Example 8

### 6-(Methylsulfonyl)-2-[4-(3-thienyl)phenyl]-1H-benzimidazole

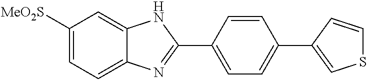

This compound was prepared from 2-(4-bromophenyl)-6-(methylsulfonyl)-1H-benzimidazole and 3-thienylboronic acid according to the procedure described in Example 3, step 4. MS (ESI, Q⁺) m/z 355 (M+1).

### Example 9

### 4′-[6-(Methylsulfonyl)-1H-benzimidazol-2-yl]biphenyl-4-ol

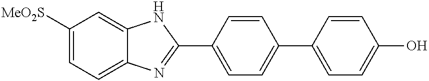

This compound was prepared from 2-(4-bromophenyl)-6-(methylsulfonyl)-1H-benzimidazole and 4-hydroxyphenylboronic acid according to the procedure described in Example 3, Step 4.

¹H NMR (400 MHz, d₆-acetone): δ 8.30 (2H, d, J=8.2 Hz), 8.24 (1H, br s), 7.84-7.77 (4H, m), 7.63 (2H, d, J=8.4 Hz), 6.97 (2H, d, J=8.4 Hz), 3.14 (3H, s).

MS (ESI, Q⁺) m/z 365 (M+1).

### Example 10

### 4′-[6-(Methylsulfonyl)-1H-benzimidazol-2-yl]biphenyl-3-ol

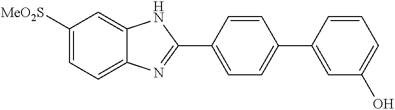

This compound was prepared from 2-(4-bromophenyl)-6-(methylsulfonyl)-1H-benzimidazole and 3-hydroxyphenylboronic acid according to the procedure described in Example 3, Step 4. MS (ESI, Q⁻) m/z 363 (M-1).

### Example 11

### 4′-[6-(Methylsulfonyl)-1H-benzimidazol-2-yl]biphenyl-2-ol

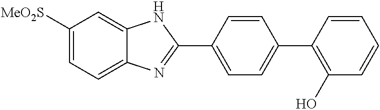

This compound was prepared from 2-(4-bromophenyl)-6-(methylsulfonyl)-1H-benzimidazole and 2-hydroxyphenylboronic acid according to the procedure described in Example 3, Step 4. MS (ESI, Q⁺) m/z 365 (M+1).

### Example 12

### 6-(Methylsulfonyl)-2-(4-pyridin-3-ylphenyl)-1H-benzimidazole

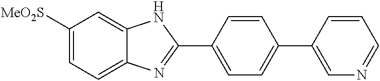

This compound was prepared from 2-(4-bromophenyl)-6-(methylsulfonyl)-1H-benzimidazole and pyridine-3-boronic acid according to the procedure described in Example 3, Step 4. MS (ESI, Q⁺) m/z 350 (M+1).

### Example 13

### 2-(3′-Chlorobiphenyl-4-yl)-6-(methylsulfonyl)-1H-benzimidazole

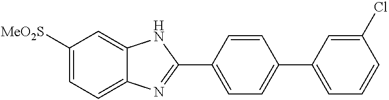

This compound was prepared from 2-(4-bromophenyl)-6-(methylsulfonyl)-1H-benzimidazole and 3-chlorophenylboronic acid according to the procedure described in Example 3, Step 4. MS (ESI, Q⁺) m/z 383 (M+1).

### Example 14

### 6-(Methylsulfonyl)-2-[4-(2-thienyl)phenyl]-1H-benzimidazole

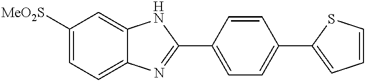

### Step 1: {4-[6-(Methylsulfonyl)-1H-benzimidazol-2-yl]phenyl}boronic acid

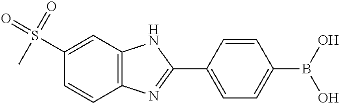

Into a flame-dried round-bottom flask equipped with a magnetic stirbar and under nitrogen was added 2-(4-bromophenyl)-6-(methylsulfonyl)-1H-benzimidazole [Example 3, step 3] (600 mg, 1.70 mmol), bis(pinacolato)diboron (650 mg, 2.55 mmol), potassium acetate (670 mg, 6.85 mmol), PdCl₂(dppf)-CH₂Cl₂adduct (70 mg, 0.05 mmol) and DMF (11 mL). The suspension was degassed with nitrogen for 20 min, before being heated at 85° C. for 24 h. The reaction mixture was cooled and the solvent was evaporated under reduced pressure. The resulting solids were diluted with ethyl acetate and poured into a 250 mL separatory funnel containing water. The mixture was extracted with ethyl acetate (3×30 mL). The combined organic layers were washed with brine, dried over MgSO₄, filtered and the solvent was evaporated under reduced pressure. The product was purified by filtering through a short pad of silica gel and washing with 70% ethyl acetate in hexanes to give the desired product as light beige solid. MS (ESI, Q⁺) m/z 399 (M+1).

### Step 2: 6-(Methylsulfonyl)-2-[4-(2-thienyl)phenyl]-1H-benzimidazole

Into a 5 mL microwave vial equipped with a magnetic stirbar was added {4-[6-(methylsulfonyl)-1H-benzimidazol-2-yl]phenyl}boronic acid (20 mg, 0.05 mmol), 2-bromothiophene (15 μL, 0.15 mmol), 2 M aqueous Na₂CO₃ solution (50 μL, 0.10 mmol), PdCl₂(dppf)-CH₂Cl₂ adduct (4 mg, 0.005 mmol) and DMF (0.5 mL). The vial was sealed and heated to 125° C. under microwave irradiation for 30 min. The reaction mixture was concentrated and purified by column chromatography on silica gel to give the desired product as a white solid.

¹H NMR (500 MHz, d₆-DMSO): δ 8.26 (2H, d, J=8.0 Hz), 8.19-8.11 (1H, br s), 7.90 (2H, d, J=8.0 Hz), 7.86-7.79 (1H, br s), 7.79-7.74 (1H, m), 7.71-7.68 (1H, m), 7.65 (1H, d, J=5.0 Hz), 7.21 (1H, t, J=4.5 Hz), 3.24 (3H, s). MS (ESI, Q⁺) m/z 355 (M+1).

### Example 15

### 2-[4-(3-Furyl)phenyl]-6-(methylsulfonyl)-1H-benzimidazole

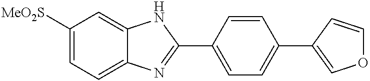

This compound was prepared from {4-[6-(methylsulfonyl)-1H-benzimidazol-2-yl]phenyl}boronic acid [Example 14, step 1] and 3-bromofuran according to the procedure described in Example 14, step 2. MS (ESI, Q⁺) m/z 339 (M+1).

### Example 16

### 6-(Methylsulfonyl)-2-[4-(3-methyl-2-thienyl)phenyl]-1H-benzimidazole

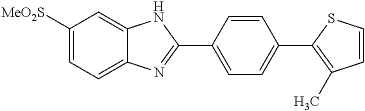

This compound was prepared from {4-[6-(methylsulfonyl)-1H-benzimidazol-2-yl]phenyl}boronic acid [Example 14, step 1] and 2-bromo-3-methylthiophene according to the procedure described in Example 14, step 2. MS (ESI, Q⁺) m/z 369 (M+1).

### Example 17

### 2-(2′-Fluorobiphenyl-4-yl)-N,N-dimethyl-1H-benzimidazole-6-sulfonamide

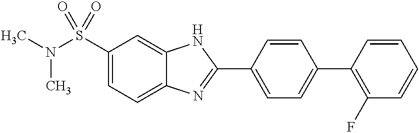

Into a 25 mL round-bottom flask equipped with a magnetic stirbar was added 3,4-diamino-N,N-dimethylbenzenesulfonamide (1.00 g, 4.7 mmol). To this was added a solution of 2′-fluorobiphenyl-4-carbaldehyde (from Example 1, step 1, 800 mg, 4.0 mmol) in DMF (10 mL) and the reaction mixture stirred at room temperature for 5 min. Chlorotrimethylsilane (1.28 mL, 10.0 mmol) was added dropwise and the reaction mixture stirred for an additional 5 min, and then heated to 90° C. in a reaction block for 16 h. The reaction mixture was cooled to room temperature and quenched with dropwise addition of 2M aqueous Na₂CO₃. The mixture was poured into a 250 mL separatory funnel containing water (75 mL) and the mixture was extracted with dichloromethane (3×50 mL). The combined organic layers were washed with brine, dried over MgSO₄, filtered and the solvent was evaporated under reduced pressure. Purification by column chromatography through silica gel, eluting with 25% EtOAc in hexanes to 100% EtOAc in hexanes as a gradient over 35 min, yielded the title compound as a foam.

MS (ESI, Q⁺) m/z 396 (M+1).

### Example 18

### 2-(2′-Fluorobiphenyl-4-yl)-6-(methylsulfonyl)-1H-imidazo[4,5-b]pyridine

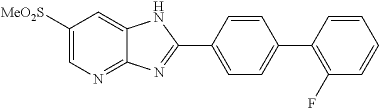

### Step 1: 5-(Methylsulfonyl)pyridine-2,3-diamine

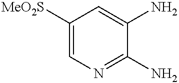

Into a 100 mL round-bottom flask equipped with a magnetic stirbar was added 2,3-diamino-5-bromopyridine (1.50 g, 8.00 mmol), methanesulfinic acid sodium salt (1.63 g, 16.0 mmol), copper (I) trifluoromethanesulfonate benzene complex (2.00 g, 4.00 mmol) and DMSO (15 mL). The suspension was treated with trans-1,2-diaminocyclohexane (0.48 mL, 4.00 mmol) and the resulting grey suspension heated to 130° C. for 16 h. The mixture was cooled and filtered through a pad of celite on a sintered glass funnel, washing with ethyl acetate. The filtrate was poured into a 500 mL separatory funnel containing 1M aqueous HCl (250 mL) and the mixture was extracted with ethyl acetate (3×75 mL). The combined organic layers were washed with brine, dried over MgSO₄, filtered and the solvent was evaporated under reduced pressure. Purification by column chromatography through silica gel, eluting with 100% EtOAc to 5% MeOH in EtOAc as a gradient over 35 min, gave the title compound as a light brown solid. MS (ESI, Q⁺) m/z 188 (M+1).

### Step 2: 2-(2′-Fluorobiphenyl-4-yl)-6-(methylsulfonyl)-1H-imidazo[4,5-b]pyridine

Into a 10 mL vial equipped with a magnetic stirbar was added 5-(methylsulfonyl)pyridine-2,3-diamine (81 mg, 0.433 mmol). To this was added a solution of 2′-fluorobiphenyl-4-carbaldehyde (from Example 1, step 1, 87 mg, 0.43 mmol) in DMF (3 mL) and the reaction mixture stirred at room temperature for 5 min. Chlorotrimethylsilane (0.14 mL, 1.08 mmol) was added dropwise and the reaction mixture stirred for an additional 5 min, and then heated to 90° C. in a reaction block for 16 h. The reaction mixture was cooled to room temperature and quenched with dropwise addition of saturated aqueous Na₂CO₃ solution. The mixture was poured into a phase separator with 10 mL of water and extracted with dichloromethane (2×5 mL). The organic layer was concentrated under vacuum and purified by reverse phase high pressure liquid chromatography through a C,₈ column.

¹H NMR (400 MHz, d₆-acetone): δ 8.90 (1H, d, J=2.0 Hz), 8.51-8.49 (3H, m), 7.85-7.82 (2H, m), 7.70-7.65 (1H, m), 7.53-7.48 (1H, m), 7.40-7.30 (2H, m), 3.30 (3H, s).

MS (ESI, Q⁺) m/z 368 (M+1).

### Example 19

### 2-[6-(2-Fluorophenyl)pyridin-3-yl]-6-(methylsulfonyl)-1H-benzimidazole

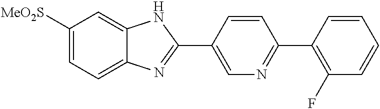

### Step 1: 6-(2-Fluorophenyl)nicotinaldehyde

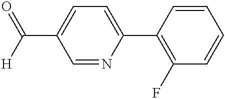

Into a 20 mL microwave vial equipped with a magnetic stirbar was added 6-bromonicotinaldehyde (1.0 g, 5.38 mmol), 2-fluorophenylboronic acid (0.9 g, 6.45 mmol), PdCl₂(dppf)-CH₂Cl₂ adduct (0.2 g, 0.27 mmol), 2 M aqueous Na₂CO₃ solution (5.4 mL, 10.8 mmol) and DMF (8 mL). The vial was sealed and heated to 125° C. under microwave irradiation for 30 min. The cooled mixture was poured into a 250 mL separatory funnel containing water and the mixture extracted with ethyl acetate (3×30 mL). The combined organic layers were washed with brine, dried over MgSO₄, filtered through a pad of silica gel and concentrated under reduced pressure. Purification by column chromatography through silica gel gave the title compound as an off-white solid.

### Step 2: 2-[6-(2-Fluorophenyl)pyridin-3-yl]-6-(methylsulfonyl)-1H-benzimidazole

This compound was prepared from 4-(methylsulfonyl)benzene-1,2-diamine [Example 3, step 2] and 6-(2-fluorophenyl)nicotinaldehyde according to the procedure described in Example 3, step 3.

¹H NMR (400 MHz, d₆-acetone): δ 9.55 (1H, d, J=2.5 Hz), 8.67 (1H, dd, J=8.5, 2.5 Hz), 8.25 (1H, s), 8.17 (1H, td, J=8.0, 2.0 Hz), 8.09-8.05 (1H, m), 7.88-7.82 (2H, m), 7.57-7.51 (1H, m), 7.40-7.27 (2H, m), 3.16 (3H, s).

MS (ESI, Q⁺) m/z 368 (M+1).

### Example 20

### 2-[5-(2-Fluorophenyl)pyridin-2-yl]-6-(methylsulfonyl-1H-benzimidazole

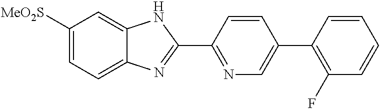

### Step 1: 5-(2-Fluorophenyl)pyridine-2-carbaldehyde

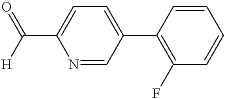

This compound was prepared from 5-bromopyridine-2-carbaldehyde and 2-fluorophenylboronic acid according to the procedure described in Example 20, step 1.

### Step 2: 2-[5-(2-Fluorophenyl)pyridin-2-yl]-6-(methylsulfonyl)-1H-benzimidazole

This compound was prepared from 4-(methylsulfonyl)benzene-1,2-diamine [Example 3, step 2] and 5-(2-fluorophenyl)pyridine-2-carbaldehyde according to the procedure described in Example 3, step 3.

MS (ESI, Q⁺) m/z 368 (M+1).

### Example 21

### 2-(2′-Fluorobiphenyl-4-yl)-1H-benzimidazole-6-sulfonamide

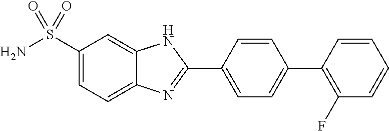

### Step 1: 2-(2′-Fluorobiphenyl-4-yl)-6-iodo-1H-benzimidazole

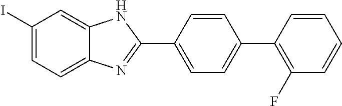

Into a 10 mL pressure tube equipped with a magnetic stirbar was added 4-iodobenzene-1,2-diamine (600 mg, 2.56 mmol), 2′-fluorobiphenyl-4-carbaldehyde (513 mg, 2.56 mmol) and DMF (4 mL). The reaction mixture was treated with drop wise addition of TMSCl (0.81 mL, 6.41 mmol), the vial sealed, and the mixture heated to 90° C. for 4 h. The cooled reaction mixture was poured into a 125 mL separatory funnel containing 2 M aqueous Na₂CO₃ solution (75 mL) and the mixture extracted with ethyl acetate (3×30 mL). The combined organic layers were washed with brine, dried over MgSO₄, filtered and concentrated under reduced pressure. Purification by column chromatography through silica gel, eluting with 5% acetone in toluene afforded the desired product as a solid.

### Step 2: 2-(2′-Fluorobiphenyl-4-yl)-6-[(tripropan-2-ylsily)sulfanyl]-1H-benzimidazole

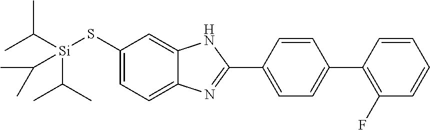

Into a 4 mL vial equipped with a magnetic stirbar was weighted 2-(2′-fluorobiphenyl-4-yl)-6-iodo-1H-benzimidazole (33 mg, 0.080 mmol), potassium tripropan-2-ylsilanethiolate (22 mg, 0.100 mmol) and PdCl₂(dppf)-CH₂Cl₂ adduct (3.3 mg, 0.004 mmol). The solids were suspended in dioxane (0.7 mL) and heated to 90° C. for 3 h. The mixture was poured into a phase separatory cartridge containing water (4 mL) and extracted with CH₂Cl₂ (3×4 mL). The combined organic layers were concentrated and purified by eluting through a short pad of silica gel using 20% EtOAc in hexanes. The resulting yellow oil, obtained after concentrated of the solvent, was used directly in the next step.

### Step 3: 2-(2′-Fluorobiphenyl-4-yl)-1H-benzimidazole-6-sulfonamide

Into a 5 mL round-bottom flask equipped with a magnetic stirbar was added 2-(2′-fluorobiphenyl-4-yl)-6-[(tripropan-2-ylsilyl)sulfanyl]-1H-benzimidazole (38 mg, 0.080 mmol) in MeCN (1 mL). The solution was cooled to 0° C. and potassium nitrate (28 mg, 0.280 mmol) was added, followed by drop wise addition of sulfuryl chloride (23 μL, 0.280 mmol). The reaction mixture was stirred at 0° C. for 2 h and then diluted with EtOAc (5 mL), filtered through a plug of celite on a sintered glass funnel, and the filtrate concentrated under reduced pressure. The crude reaction mixture was diluted with THF (1 mL) and cooled to 0° C. An excess of concentrated aqueous ammonium hydroxide (54 μL, 0.800 mmol) was added drop wise and the mixture stirred at this temperature for 0.5 h. The reaction mixture was poured into a 25 mL separatory funnel containing saturated aqueous NH₄Cl solution (10 mL) and extracted with EtOAc (3×5 mL). The combined organic layers were washed with brine, dried over MgSO₄, filtered and concentrated under reduced pressure. Purification by column chromatography through silica gel, eluting with 30-90% EtOAc in hexanes as a gradient, afforded the title compound as a beige solid. MS (ESI, Q⁺) m/z 368 (M+1).

### Example 22

### 2-(2′-Fluorobiphenyl-4-yl)-6-(methylsulfanyl)-1H-benzimidazole

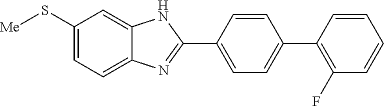

### Step 1: 5-(Methylsulfanyl)-2-nitroaniline

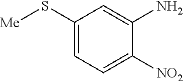

Into a 250 mL round-bottom flask equipped with a magnetic stirbar was added 5-chloro-2-nitroaniline (15.0 g, 87.0 mmol) and DMF (125 mL). The yellow-orange solution was treated with portion wise addition of sodium thiomethoxide (9.75 g, 139.0 mmol) and the resulting mixture was heated to 65° C. for 20 h. The mixture was cooled to room temperature and poured into a 500 mL separatory funnel containing water (250 mL) and extracted with ethyl acetate (3×75 mL). The combined organic layers were washed with brine, dried over MgSO₄, filtered and the solvent was evaporated under reduced pressure. Purification by column chromatography through silica gel, eluting with 0% EtOAc in hexanes to 50% EtOAc in hexanes as a gradient afforded the title compound as an orange solid.

### Step 2: 4-(Methylsulfanyl)benzene-1,2-diamine

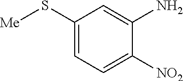

Into a 250 mL round-bottom flask equipped with a magnetic stirbar was added 5-(methylsulfanyl)-2-nitroaniline (2.50 g, 13.6 mmol) and ethanol (100 mL). The solution was treated with tin(II)chloride dihydrate (15.3 g, 67.9 mmol) and the reaction mixture was refluxed for 16 h. The cooled reaction mixture was concentrated under reduced pressure to 25 mL and the solution was basified with 1 M aqueous NaOH solution (approx 100 mL) to pH=10. The resulting milky beige suspension was poured into a 250 mL separatory funnel and the mixture was extracted with ethyl acetate (3×50 mL). The combined organic layers were washed with brine, dried over MgSO₄, filtered and the solvent was evaporated under reduced pressure.

### Step 3: 2-(2′-Fluorobiphenyl-4-yl)-6-(methylsulfanyl)-1H-benzimidazole

Into a 10 mL pressure vial equipped with a magnetic stirbar was added 4-(methylsulfanyl)benzene-1,2-diamine (289 mg, 1.873 mmol), 2′-fluorobiphenyl-4-carbaldehyde (250 mg, 1.25 mmol) and DMF (3 mL). The solution was treated with TMSCl (0.40 mL, 3.12 mmol) and stirred at room temperature for 10 minutes before being heated to 90° C. in an oil bath for 2 h. The cooled reaction mixture was quenched with drop wise addition of 2 M aqueous Na₂CO₃ solution (5 mL). The mixture was diluted with dichloromethane (5 mL) and the phases separated using a phase extractor. Additional water was added and the mixture was further extracted with dichloromethane (3×10 mL). The combined organic layers were concentrated under reduced pressure. Purification by column chromatography through silica gel, eluting with 0% EtOAc in hexanes to 60% EtOAc in hexanes as a gradient, afforded the title compound as off-white solid. Further purification by trituration in acetone and filtering through filter paper on a Hirsch funnel, gave an off-white solid. MS (ESI, Q⁺) m/z 335 (M+1).

### Example 23

### 2-(2′-Fluorobiphenyl-4-yl)-6-(methylsulfinyl)-1H-benzimidazole

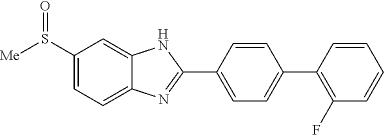

Into a 250 mL round-bottom flask equipped with a magnetic stirbar was added 2-(2′-Fluorobiphenyl-4-yl)-6-(methylsulfanyl)-1H-benzimidazole (120 mg, 0.36 mmol) and methanol (2 mL). The solution was cooled to 0° C. in an ice bath and sodium periodate (84 mg, 0.40 mmol) in water (1 mL) was added portion wise over 30 minutes. The suspension was stirred at 0° C. for 2 h and then allowed to warm to room temperature over 16 h. The reaction mixture was concentrated to remove the methanol and the orange suspension was poured into a 75 mL separatory funnel containing water (30 mL) and the mixture was extracted with ethyl acetate (3×25 mL). The combined organic layers were washed with brine, dried over MgSO₄, filtered and the solvent was evaporated under reduced pressure. Purification by column chromatography through silica gel, eluting with 100% EtOAc to 20% EtOH in EtOAc as a gradient afforded the desired product as a beige solid. MS (ESI, Q⁺) m/z 351 (M+1).

### Example 24

### 6-(Methylsulfonyl)-2-(6-phenylpyridazin-3-yl)-1H-benzimidazole

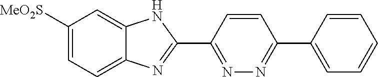

### Step 1: 3-Ethenyl-6-phenylpyridazine

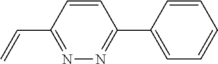

Into a 250 mL round-bottom flask equipped with a magnetic stirbar was weighted 3-chloro-6-phenyl pyridazine (5.00 g, 26.2 mmol), bis(triphenylphosphine)palladium chloride (0.921 g, 1.31 mmol), and LiCl (5.56 g, 131 mmol). The flask was evacuated under vacuum (1 mm Hg) and backfilled with N₂ (repeated 3 times). The solids were suspended in DMF (75 mL) and N₂ gas was bubbled into the solution via syringe for 20 minutes. After this time tetravinyltin (7.68 mL, 42.0 mmol) was added and the suspension heated to 100° C. for 16 h. The reaction mixture was filtered through a plug of silica gel on sintered glass funnel, washing with diethyl ether. The filtrate was poured into a 250 mL separatory funnel containing water (125 mL) and extracted with diethyl ether (3×50 mL). The combined organic layers were washed with brine, dried over MgSO₄, filtered and the solvent was evaporated under reduced pressure. Purification by column chromatography through silica gel, eluting with 0% EtOAc in hexanes to 40% EtOAc in hexanes as a gradient, afforded the desired product as an off-white solid.

### Step 2: 6-Phenylpyridazine-3-carbaldehyde

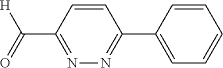

Into a 100 mL round-bottom flask equipped with a magnetic stirbar was added 3-ethenyl-6-phenylpyridazine (300 mg, 1.646 mmol) in dichloromethane (10 mL). The solution was cooled to −78° C. and then ozone was bubbling in through a gas dispersion tube for 1.5 h until the solution turned a slight blue colour. Ozone bubbling was stopped and the reaction mixture was stirred at −78° C. for 30 minutes and then quenched with dimethyl sulfide (1.22 mL, 16.5 mmol). The solution was allowed to warm to room temperature for 1 h and then concentrated under reduced pressure. The crude residue was poured into a 125 mL separatory funnel containing saturated aqueous NaHCO₃ solution (75 mL) and extracted with ethyl acetate (3×30 mL). The combined organic layers were washed with brine, dried over MgSO₄, filtered and the solvent was evaporated under reduced pressure.

Purification by column chromatography through silica gel, eluting with 0% EtOAc in hexanes to 50% EtOAc in hexanes as a gradient, afforded the title compound as a light yellow solid. MS (ESI, Q⁺) m/z 185 (M+1).

### Step 3: 6-(Methylsulfonyl)-2-(6-phenylpyridazin-3-yl)-1H-benzimidazole

Into a 10 mL vial equipped with a magnetic stirbar was weighted 4-(methylsulfonyl)benzene-1,2-diamine (121 mg, 0.65 mmol). To this was added a solution of 6-phenylpyridazine-3-carbaldehyde (80 mg, 0.434 mmol) in DMF (3 mL) and the reaction mixture stirred at room temperature for 5 minutes. TMSCl (0.14 mL, 1.09 mmol) was added drop wise, and the reaction mixture was heated to 90° C. in a reaction block, exposed to the atmosphere. Heating was continued for 16 h. The reaction mixture was quenched with drop wise addition of a saturated aqueous Na₂CO₃ solution (5 mL). The mixture was poured into a 125 mL separatory funnel containing water (75 mL) and extracted with ethyl acetate (3×30 mL). The combined organic layers were washed with brine, dried over MgSO₄, filtered and the solvent was evaporated under reduced pressure. Purification by column chromatography through silica gel, eluting with 50% EtOAc in hexanes to 100% EtOAc in hexanes as a gradient, afforded a white solid. The indicated compound was further purified by trituration in acetone. MS (ESI, Q⁺) m/z 351 (M+1).

### Example 25

### 6-(Methylsulfonyl)-2-(2-phenylpyrimidin-5-yl)-1H-benzimidazole

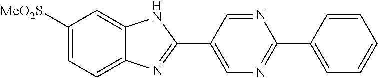

### Step 1: 2-Phenylpyrimidine-5-carbaldehyde

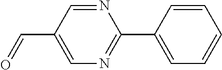

Into a 100 mL round-bottom flask equipped with a magnetic stirbar was added 2-phenyl-5-methanolpyrimidine (1.00 g, 5.4 mmol) and sodium bicarbonate (0.90 g, 10.7 mmol) in dichloromethane (20 mL). The suspension was treated with portion wise addition of Dess-Martin periodinane (2.73 g, 6.44 mmol) and the white suspension was stirred at room temperature for 1 h. The reaction mixture was filtered through a plug of celite on a sintered glass funnel, washing with ethyl acetate. The filtrate was poured into a 250 mL separatory funnel containing water (125 mL) and the mixture was extracted with ethyl acetate (3×30 mL). The combined organic layers were washed with brine, dried over MgSO₄, filtered and the solvent was evaporated under reduced pressure.

Purification by column chromatography through silica gel, eluting with 0% EtOAc in hexanes to 50% EtOAc in hexanes as a gradient, afforded the desired product as a white solid. MS (ESI, Q⁺) m/z 185 (M+1).

### Step 2: 6-(Methylsulfonyl)-2-(2-phenylpyrimidin-5-yl)-1H-benzimidazole

Into a 10 mL vial equipped with a magnetic stirbar was weighted 4-(methylsulfonyl)benzene-1,2-diamine (303 mg, 1.63 mmol) 2-phenylpyrimidine-5-carbaldehyde (250 mg, 1.36 mmol) and DMF (3 mL). The reaction mixture stirred at room temperature while TMSCl (0.43 mL, 3.39 mmol) was added drop wise over 5 minutes, and then heated to 90° C. in a reaction block, exposed to the atmosphere. Heating was continued for 4 h, and then the mixture was stirred for 16 h at room temperature. The reaction was quenched with drop wise addition of 2 M aqueous Na₂CO₃ solution (5 mL) and poured into a phase separator with 10 mL of water, and extracted with dichloromethane (5 mL). The organic layers were concentrated under reduced pressure and purified by column chromatography through silica gel, eluting with 5% EtOAc in hexanes to 50% EtOAc in hexanes as a gradient. The product was further purified by trituration in ethyl acetate, filtering through filter paper on a Hirsch funnel, and washing with diethyl ether to give an off-white solid. MS (ESI, Q⁺) m/z 351 (M+1).

### Example 26

### 2-(3′-Fluorobiphenyl-4-yl)-7-(methylsulfonyl)imidazo[1,2-a]pyridine

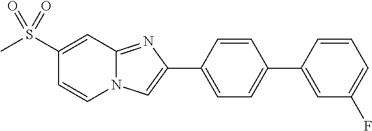

### Step 1: 4-(Methylthio)pyridin-2-amine

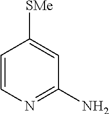

A mixture of 4-chloropyridin-2-amine (3.00 g, 23.3 mmol) and sodium thiomethoxide (4.9 g, 70.0 mmol) in ethanol (37.3 mL) and water (9.3 mL) was heated at 140° C. in a sealed tube for 18 h. The solvent was evaporated and the residue was triturated with water (20 mL) and Et₂O (5 mL). The mixture was filtered and washed with water followed by Et₂O. The solid was dried under high vacuum to afford the title product. MS (ESI, Q⁺) m/z 141 (M+1).

### Step 2: 2-(4-Bromophenyl)-7-(methylthio)imidazo[1,2-a]pyridine

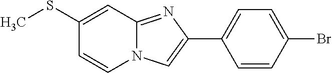

A mixture of 4-(methylthio)pyridin-2-amine (1.00 g, 7.1 mmol) and 2-bromo-1-(4-bromophenyl)ethanone (2.20 g, 7.6 mmol) in ethanol (71 mL) was heated at 100° C. under reflux for 8 h. The mixture was diluted with saturated aqueous NaHCO₃ solution (40 mL) and heated again at 100° C. for 1 h. The mixture was cooled to room temperature and diluted with water (200 mL). The solid was filtered and washed with water followed by Et₂O. The solid was dried under high vacuum to afford the title product. MS (ESI, Q⁺) m/z 319, 321 (M+1 for ⁷⁹Br and ⁸¹Br).

### Step 3: 2-(4-Bromophenyl)-7-(methylsulfonyl)imidazo[1,2-a]pyridine

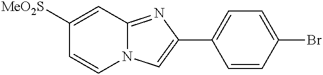

To a solution of 2-(4-bromophenyl)-7-(methylthio)imidazo[1,2-a]pyridine (1.90 g, 5.8 mmol) in acetic acid (29 mL) was added sodium tungstate dihydrate (0.19 g, 0.58 mmol) followed by hydrogen peroxide (2.0 mL, 23.2 mmol) and the mixture was stirred at room temperature for 3 h. The solvent was evaporated, the residue was mixed with saturated aqueous NaHCO₃ solution (30 mL) and Et₂O (10 mL). The mixture was filtered and washed with water followed by Et₂O. The crude product was recrystallized from MeOH/Et₂O, filtered and washed with Et₂O to afford the title product.

### Step 4: 2-(3′-Fluorobiphenyl-4-yl)-7-(methylsulfonyl)imidazo[1,2-a]pyridine

A mixture of 2-(4-bromophenyl)-7-(methylsulfonyl)imidazo[1,2-a]pyridine (50 mg, 0.14 mmol), (3-fluorophenyl)boronic acid (30 mg, 0.21 mmol), 1,1′-bis(diphenylphosphino)ferrocene-palladium(II)dichloride dichloromethane complex (11 mg, 0.014 mmol) in degassed sodium carbonate (0.28 mL, 0.29 mmol) and dioxane (1.4 mL) was heated at 90° C. for 18 h. The mixture was filtered through a pad of silica gel eluting with EtOAc. The solvent was evaporated and the mixture was recrystallized from acetone/Et₂O, filtered and washed with Et₂O to afford the title product.

¹H NMR (500 MHz, d₆-acetone): δ 8.78 (1H, d, J=7.0 Hz), 8.65 (1H, s), 8.22 (2H, d, J=8.0 Hz), 8.16 (1H, s), 7.84 (2H, d, J=8.0 Hz), 7.61 (1H, d, J=7.5 Hz), 7.58-7.51 (2H, m), 7.37 (1H, d, J=7.0 Hz), 7.24-7.12 (1H, m), 3.28 (3H, s).

MS (ESI, Q⁺) m/z 367 (M+1).

### Example 27

### 2-(2′-Fluorobiphenyl-4-yl)-7-(methylsulfonyl)imidazo[1,2-a]pyridine

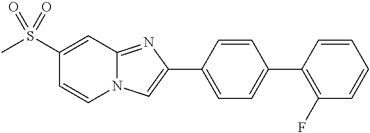

The title compound was prepared in a similar manner as described for Example 26, Step 6 from 2-(4-bromophenyl)-7-(methylsulfonyl)imidazo[1,2-a]pyridine and (2-fluorophenyl)boronic acid. MS (ESI, Q⁺) m/z 367 (M+1).

### Example 28

### 2-(4′-Fluorobiphenyl-4-yl)-7-(methylsulfonyl)imidazo[1,2-a]pyridine

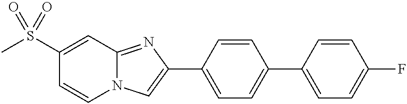

The title compound was prepared in a similar manner as described for Example 26, Step 6 from 2-(4-bromophenyl)-7-(methylsulfonyl)imidazo[1,2-a]pyridine and (4-fluorophenyl)boronic acid. MS (ESI, Q⁺) m/z 367 (M+1).

### Example 29

### 7-(Methylsulfonyl)-2-[3′-(trifluoromethyl)biphenyl-4-yl]imidazo[1,2-a]pyridine

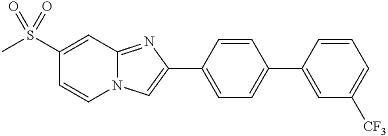

The title compound was prepared in a similar manner as described for Example 26, Step 6 from 2-(4-bromophenyl)-7-(methylsulfonyl)imidazo[1,2-a]pyridine and [3-(trifluoromethyl)phenyl]boronic acid. MS (ESI, Q⁺) m/z 417 (M+1).

### Example 30

### 2-(3′-Chlorobiphenyl-4-yl)-7-(methylsulfonyl)imidazo[1,2-a]pyridinec

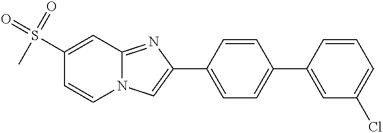

The title compound was prepared in a similar manner as described for Example 26, Step 6 from 2-(4-bromophenyl)-7-(methylsulfonyl)imidazo[1,2-a]pyridine and (3-chlorophenyl)boronic acid. MS (ESI, Q⁺) m/z 383 (M+1).

### Example of a Pharmaceutical Formulation

As a specific embodiment of an oral pharmaceutical composition of the present invention, a 100 mg potency tablet is composed of 100 mg of any one of Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 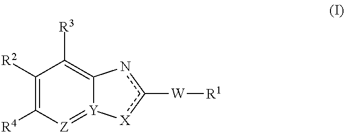
or a pharmaceutically acceptable salt thereof; wherein
X is NH, Y is C, and Z is N or CR⁵;
or X and Z are each CR⁵, and Y is N;
W is a residue selected from the group consisting of: 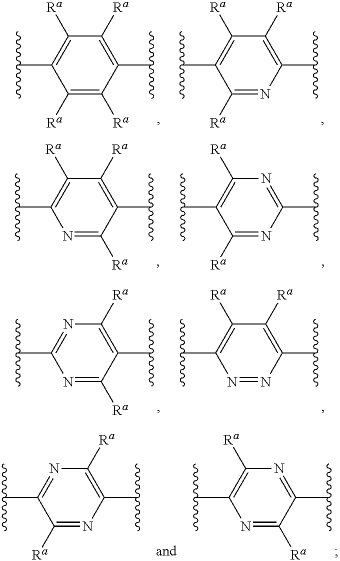
wherein each R^{a} is independently selected from the group consisting of:
hydrogen,
halogen,
C₁₋₄ alkyl, optionally substituted with one to five fluorines, and
C₁₋₄ alkoxy, optionally substituted with one to five fluorines;
R³, R⁴, and each R⁵ are each independently selected from the group consisting of:
hydrogen,
halogen,
C₁₋₄ alkyl, optionally substituted with one to five fluorines, and
C₁₋₄ alkoxy, optionally substituted with one to five fluorines;
R² is selected from the group consisting of:
—SO₂cyclopropyl,
—SC ₁₋₃ alkyl, optionally substituted with one to five fluorines,
—S(O)C₁₋₃ alkyl, optionally substituted with one to five fluorines,
—SO₂C₁₋₃ alkyl, optionally substituted with one to five fluorines, and
—SO₂NR^{b}R^{b}, wherein each R^{b} is independently hydrogen or C₁₋₃ alkyl; and
R¹ is selected from the group consisting of:
cyclopentenyl,
cyclohexenyl,
phenyl, and
heteroaryl selected from the group consisting of:
pyridyl,
pyrimidinyl,
pyridazinyl,
pyrazinyl,
furyl,
thienyl,
thiazolyl,
oxazolyl,
isoxazolyl,
isothiazolyl,
imidazolyl, and
pyrazolyl;
wherein aryl and heteroaryl are optionally substituted with one to three substituents independently selected from the group consisting of halogen, hydroxy, cyano, and C₁₋₃ alkyl wherein alkyl is optionally substituted with one to five fluorines.

2. The compound of claim 1 of structural formula (Ia) wherein X is NH, Y is C, and Z is N or CR⁵: 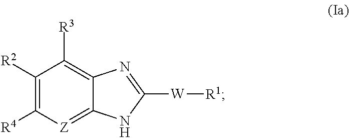
or a pharmaceutically acceptable salt thereof.

3. The compound of claim 2 wherein Z is CR₅; and R² is —SO₂CH₃ or —SO₂cyclopropyl.

4. The compound of claim 3 wherein R³, R⁴, and R⁵ are each hydrogen.

5. The compound of claim 2 wherein W is

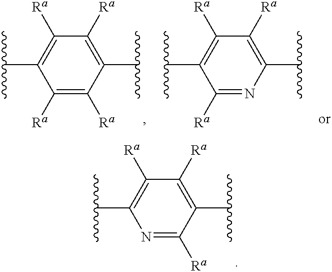

6. The compound of claim 5 wherein each R^{a} is hydrogen.

7. The compound of claim 5 wherein W is

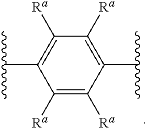

8. The compound of claim 7 wherein each R^{a} is hydrogen.

9. The compound of claim 2 wherein R¹ is phenyl optionally substituted with one to two halogens independently selected from fluorine and chlorine.

10. The compound of claim 2 wherein Z is CR⁵;
W is 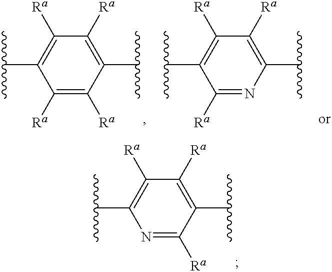
R¹ is phenyl optionally substituted with one to two halogens independently selected from chlorine and fluorine; and
R² is —SO₂CH₃ or —SO₂cyclopropyl.

11. The compound of claim 10 wherein R^{a}, R³, R⁴, and R⁵ are each hydrogen.

12. The compound of claim 1 of structural formula (Ib) wherein X and Z are each CR⁵, and Y is N: 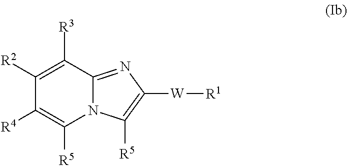
or a pharmaceutically acceptable salt thereof.

13. The compound of claim 12 wherein R² is —SO₂CH₃ or —SO₂cyclopropyl.

14. The compound of claim 13 wherein R³, R⁴, and R⁵ are each hydrogen.

15. The compound of claim 12 wherein W is

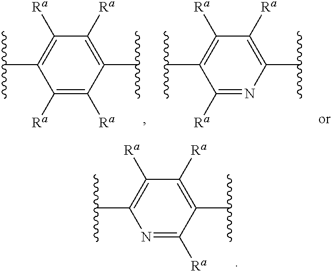

16. The compound of claim 15 wherein each R^{a} is hydrogen.

17. The compound of claim 15 wherein W is

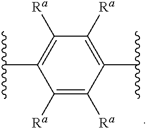

18. The compound of claim 17 wherein each R^{a} is hydrogen.

19. The compound of claim 12 wherein R¹ is phenyl optionally substituted with one to two halogens independently selected from fluorine and chlorine.

20. The compound of claim 12 wherein:
W is 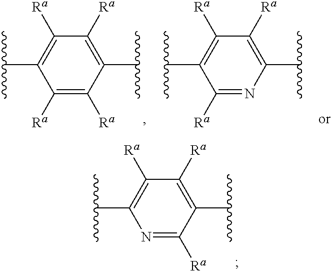
R¹ is phenyl optionally substituted with one to two halogens independently selected from chlorine and fluorine; and
R² is —SO₂CH₃ or —SO₂cyclopropyl.

21. The compound of claim 20 wherein R^{a}, R³, R⁴, and R⁵ are each hydrogen.

22. A pharmaceutical composition comprising a compound in accordance with claim 1 in combination with a pharmaceutically acceptable carrier.

23-27. (canceled)

28. A method of treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

29. The method of claim 28 wherein said lipid disorder is selected from the group consisting of dyslipidemia, hyperlipidemia, hypertriglyceridemia, atherosclerosis, hypercholesterolemia, low HDL, and high LDL.
